(19) United States
(12) Patent Application Publication
(10) Pub. No.: US 2016/0146800 A1
(43) Pub. Date: May 26, 2016

(54) APERTURE ARRAY SUBSTRATE DEVICE, A DETECTION SYSTEM AND A METHOD FOR DETECTING ANALYTES IN A SAMPLE

(71) Applicant: DUBLIN CITY UNIVERSITY, Dublin (IE)

(72) Inventors: Stephen O'Driscoll, Dublin (IE); Lourdes Basabe-Desmont, Cartagena (ES); Antonio J. Ricco, Los Gatos, CA (US); Niamh Gilmartin, Dublin (IE); Martin Somers, Carlow (IE)

(21) Appl. No.: 14/900,972

(22) PCT Filed: Jun. 24, 2014
(86) PCT No.: PCT/EP2014/063245

§ 371 Date: Dec. 22, 2015

(30) Foreign Application Priority Data
Jun. 24, 2013 (GB) 1311192.7

(51) Int. Cl. G01N 33/543; G01J 1/44; G01N 21/75; G02B 21/36

(52) U.S. Cl. CPC G01N 33/54366; G02B 21/361; G01J 1/44; G01N 21/75; G01N 33/54306; G01N 2201/062; G01N 2201/12

(57) ABSTRACT

There is provided an optical detection system (100) comprising a photodetector (110) and a substrate (120). The substrate (120) comprises an aperture array (121) wherein light is transmittable to the photodetector (110) via the apertures (122) of the substrate (120) only, and the apertures (122) are functionalised to provide capture of a target analyte (151) at an aperture such that capture of an analyte at an aperture causes attenuation of light at said aperture. The photodetector (110) being configured to detect the capture of an analyte at an aperture of the aperture array by detecting attenuation at an aperture. The system comprises a lensless detection system.

### FIELD

The present application relates to a system and method in particular an optical detection system for detection of analytes based on the use of an aperture array substrate chip device.

### BACKGROUND OF THE INVENTION

There is a need for an improved detection system and method for the detection of target analytes in a sample. In one approach the presence of an analyte in a sample may be detected using an optical system to image an analyte. However, often such systems require complex optics and complex image processing features.

The invention as described in the present specification is directed to addressing these and other problems. In particular the invention is directed to providing an improved optical detection system.

### SUMMARY

According to the present specification there is provided an optical detection system comprising a photodetector and a substrate, wherein
the substrate comprises an aperture array wherein light is transmittable to the photodetector via the apertures of the substrate only, and
the apertures are functionalised to provide capture of a target analyte at an aperture such that capture of an analyte at an aperture causes attenuation of light at said aperture,
the photodetector being configured to detect the capture of an analyte at an aperture of the aperture array by detecting attenuation at an aperture.

According to the present specification there is provided an optical detection system comprising a photodetector and a substrate in accordance with claim 1, wherein
the substrate comprises an aperture array wherein light is transmittable to the photodetector via the apertures of the substrate only, and
the apertures are functionalised to provide capture of a target analyte at an aperture such that capture of an analyte at an aperture causes attenuation of light at said aperture,
the photodetector being configured to detect the capture of an analyte at an aperture of the aperture array by detecting attenuation at an aperture,
wherein the system comprises a lensless system.

The system of the invention advantageously comprises a lensless system which obviates the requirement for complex optics and maintenance of complex optics. The system is a relatively simplified system based on the detection of attenuation of light transmitted at an aperture. The system provides a point of care detection device.

Advantageous features are provided in accordance with the dependent claims.

According to a further aspect, there is provided a substrate in accordance with claim 30 comprising a substrate configured to provide detection of capture events on the surface thereof comprising a non-transparent substrate patterned with an array of transparent apertures wherein the apertures are functionalised to provide capture of a target analyte at an aperture, wherein
the substrate patterned with the array of apertures defines a photomask, light being transmittable only through said apertures,
the size of an aperture being optimised to provide capture of a single target analyte at an aperture,
the substrate comprising a polymer substrate

Further advantageous features are provided in accordance with the dependent claims.

According to a further aspect, there is provided a substrate or an aperture array substrate device configured to provide detection of capture events on the surface thereof comprising a non-transparent substrate patterned with an array of transparent apertures wherein the apertures are functionalised to provide capture of a target analyte at an aperture.

Advantageous features are provided in accordance with the dependent claims.

According to a further aspect, there is provided a method in accordance with claim 37 for detecting the presence of a target analyte in a sample using a system according to the invention, the method comprising
Providing a substrate according to the invention;
Providing a sample to the substrate;
Passing light through the substrate to a photodetector;
Detecting light transmitted via the substrate to a photodetector;
   wherein a change in the detected light indicates presence of a target analyte.

Advantageous features are provided in accordance with the dependent claims

### BRIEF DESCRIPTION OF THE DRAWINGS

The present application will now be described with reference to the accompanying drawings in which:

FIG. 1A is a block diagram of a system according to an embodiment of the present specification;

FIG. 1B is a block diagram indicating a method of detecting a target analyte in a sample according to an embodiment of the present specification; FIGS. 1C and 1D are schematic representations of image of an exemplary aperture array chip according to an embodiment of the present specification;

FIG. 2 is a schematic representation of a microaperture array based detection system according to an embodiment of the present specification;

FIG. 3 shows illustrative images of 15 μm aperture arrays captured using a lensless optical detection system according to an embodiment of the present specification;

FIG. 4 is an illustration of the relationship between analyte capture on a microaperture array according to an embodiment of the present specification and illustrative images of the microaperture array (μAA) as captured using an optical detection system according to an embodiment of the present specification. FIGS. 4A, 4B and 4C are a sequence of images showing that as a sample flows over the μAA more analytes are captured occupying more apertures in the array. Once captured, these analytes reduce (or block completely) light arriving at the pixels below;

FIGS. 5A and 5B are images illustrating a sample system according to an embodiment of the present specification in side and perspective views respectively—the system is compact and highly portable.

FIGS. 6(a) and 6(b) are illustrative images of a microaperture array (μAA) according to an embodiment of the present application obtained using alternative image sensing means 6(a) is by conventional optical microscope and 6(b) by a lensless system according to an embodiment of the specification. The occlusion of apertures is clearly evident in both images; and

FIGS. 7(a) and 7(b) are images of Biotin functionalised beads captured at 20 μm apertures.

FIGS. 8 (a), 8(b) and 8(c) are photographs which shows an example of a real image of microaperture array (μAA) taken by an optical microscope (a) and taken by the CCD sensor (b, c). Image (b) is the raw image, and (c) is the same image after it has been processed to show better contrast. This is an empty microaperture array (μAA).

FIG. 9 shows a view of an exemplary software system according to an arrangement of the present specification. A data processing system is provided having a data acquisition module and a post-processing module for obtaining and interrogating data.

FIG. 10A (left hand image) shows an image that occurs from binding events on the microaperture array, as detected by a CMOS image sensor. The generated output, shown in FIG. 10 B (the right hand image), is produced via a series of image processing steps, including for example: (1) image enhancement, (2) image thresholding, (3) image blob analysis and (4) quantification of binding (e.g. determining number of blocked apertures). The data generated from this analysis is then processed for example using the software of FIG. 9 to provide the end-user with a percentage occupancy/binding, in this case for an array of >1000 apertures.

FIG. 11 is a graph demonstrating Percentage occupancy—the number of detected bound objects (Y axis) plotted against occlusion (percentage of apertures occluded) (X axis). Ideal is representative of results if the number of objects is equally-spaced, where an ideal is a 4-pixel deviation from the centroid.

FIG. 12 is a graph showing that a Microaperture Array System according to the present specification may be used to detect and evaluate binding events. In experimental setup, apertures of 20 μm (in diameter) were provided together with streptavidin-labelled magnetic beads (15 μm in diameter) which bind to different concentrations of biotinylated horseradish peroxidase (HRP). The number of blocked apertures is shown on the Y axis, with the concentration of biotinylated HRP shown on the X axis.

FIG. 13 shows a graph demonstrating percentage of binding events vs. occlusion. The arrangements according to the present specification provides for measuring the resultant binding/occlusion.

FIG. 14 is a graph showing percentage of binding events vs concentration for an Immuno Assay.

### DETAILED DESCRIPTION OF THE DRAWINGS

The specification provides a substrate device and a system for the detection of target analytes in a sample. In particular, the specification provides a system for the transduction and detection of biochemical binding events on the surface of a biochip. The system is an optical detection system. The specification also provides a biochip or substrate and a method of detection.

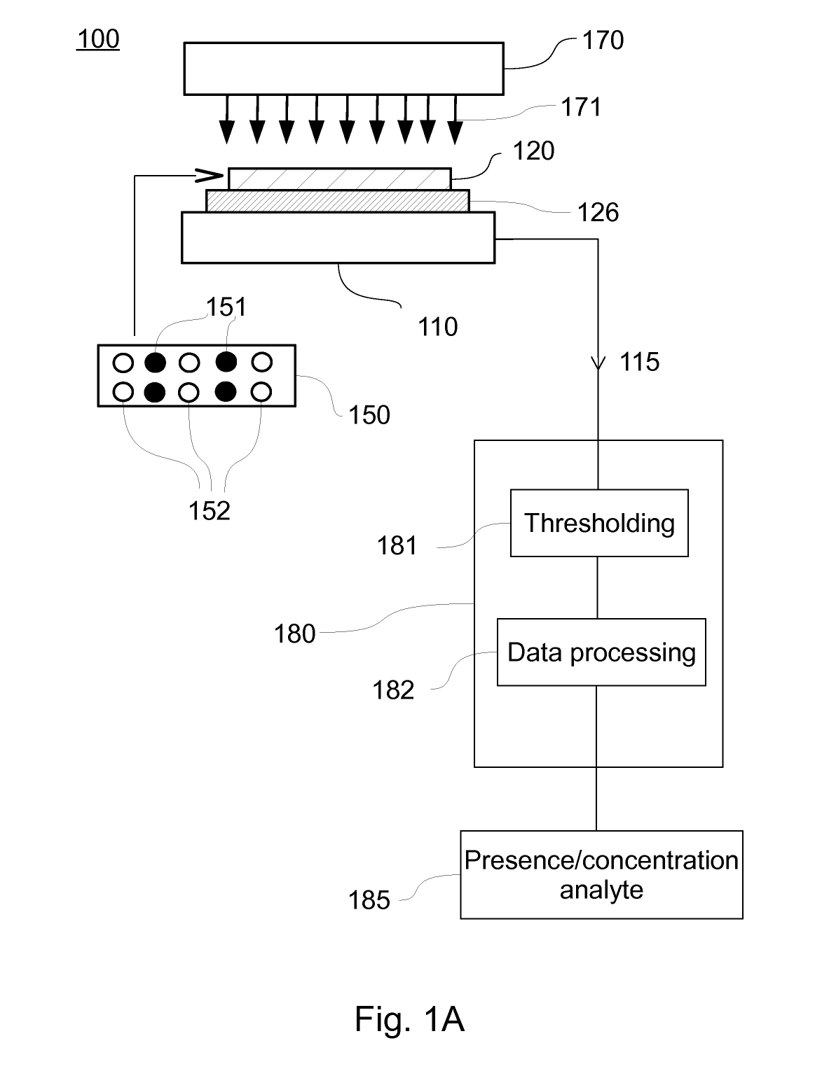

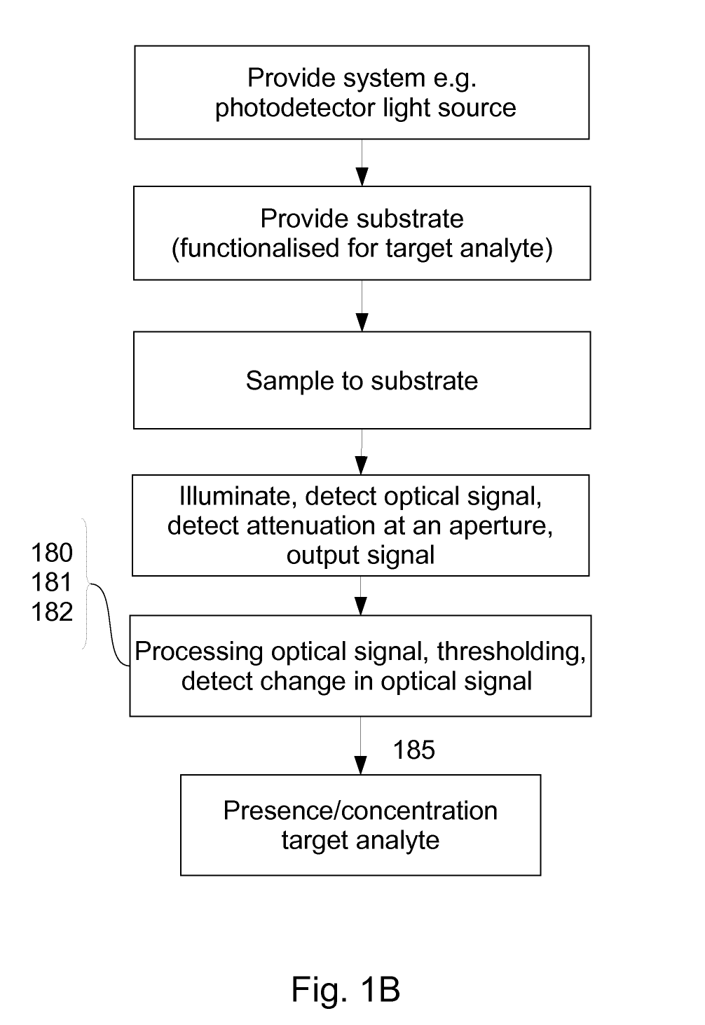

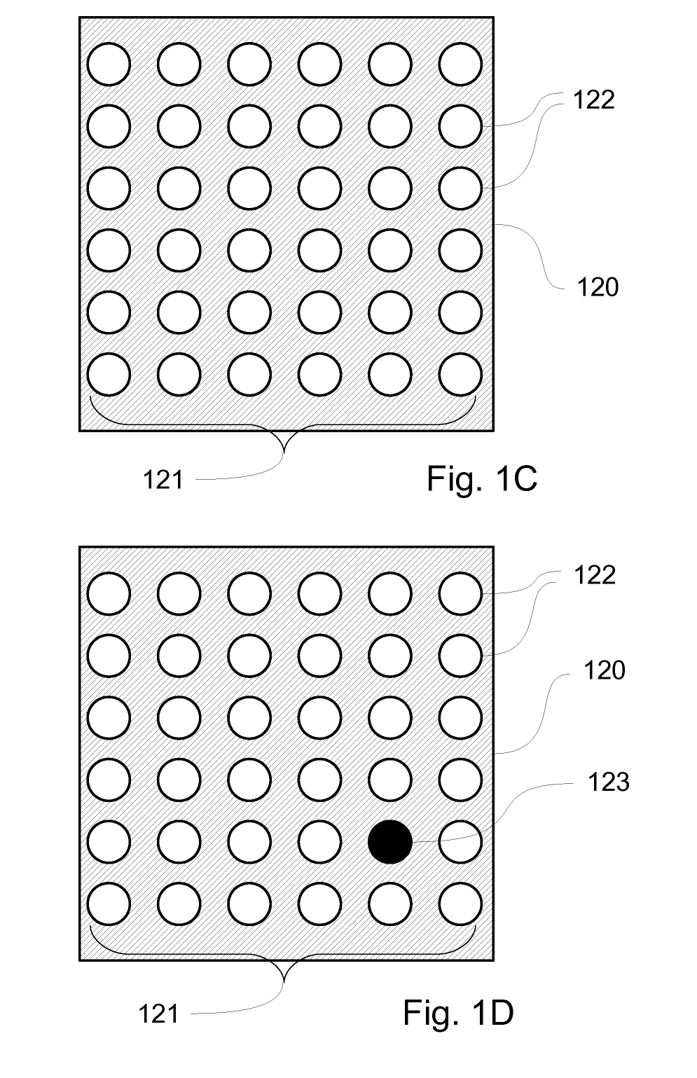

Referring to the drawings and initially in particular FIGS. 1A, 1B, 1C and 1D an exemplary system and method of an embodiment of the present specification is described.

The system 100 comprises an optical detection system including a photodetector 110. The system includes substrate or biochip 120 which comprises an aperture array 121 of apertures 122. The system further includes a light source 170 providing light 171 incident on the substrate 120. The substrate 120 is arranged such that light is transmittable to the photodetector 110 via the apertures 122 of the substrate 120 only. Apertures 122 are configured or functionalised to provide capture of a target analyte/s 151/152 of a sample 150 at an aperture 122. The capture of an analyte 151/152 at an aperture 122 causes attenuation of light at the aperture 122. The photodetector 110 is configured to detect the capture of an analyte/s 151, 152 of a sample 150 at an aperture 122 of the aperture array 121 by detecting attenuation at an aperture 122.

It is noted that while the arrangement of apertures 122 is described as an aperture array 121, the terms aperture array are not intended to be limiting. It will be appreciated that while the exemplary aperture array illustrated for example in FIG. 1C has a particular 2-dimensional (2-d) form, an aperture array may have various suitable forms. The aperture array may comprise one or more apertures. The aperture array may comprise a linear form, or another 2-d array form. It will further be appreciated that while in the exemplary arrangement of the drawings, the array is shown to be comprised of substantially circular form apertures, apertures of any suitable form may be provided. While in the example described the apertures are defined by diameter, such exemplary dimension is not intended as limiting. The dimensions of diameter are intended also to be indicative of area. Apertures of square, circular or other form of similar area dimensions may also be provided. It will further be appreciated that the number of apertures and form of apertures of an array may be selected based on properties of the type of analytes to be detected and also depending on the sensitivity required and the arrangement of the photodetector. The apertures preferably have a diameter of the order of microns. The substrate is patterned with an array of microapertures providing a microaperture array (μAA). Apertures are described herein also as microapertures, an array of apertures provided on the substrate is termed an aperture array or a microaperture array (μAA), the terms are used essentially interchangeably. Further it is noted that the terms substrate and biochip have been used interchangeably in the present specification.

It will be appreciated that the terms functionalised to provide for capture of a target analyte are not intended to be limiting. Apertures may be configured or functionalised in different ways to provide for capture of a target analyte at an aperture. For example, the configuring may include a mechanical, magnetic, or other chemical functionalisation. Further, the present specification provides for functionalisation of different apertures for capturing different analytes. Functionalisation may for example be by any chemical patterning technique, such as ink jet printing, micro-contact printing, dip pen nanolithography, or light directed synthesis.

Different apertures 122 of the array 121 may be functionalised for capture of different target analytes 151, 152 if appropriate allowing for a multiplex detection on a single substrate or biochip 120.

The substrate 120 may be a substantially non-transparent substrate patterned with an array of transparent apertures 122. However, it will be appreciated that suitable alternative substrate/aperture arrangements may be provided such that the apertures may be detected relative to the substrate.

Substrate 120 comprising the aperture array 121 defines a photomask 125, wherein light is transmittable only via said apertures 122 of the substrate.

The size of an aperture 122 of the substrate 120 is optimised to provide capture of a target analyte 151,152 at an aperture 122. Preferably the size of an aperture 122 is optimised to provide capture of a single target analyte 151,152 at the aperture. To provide the required sensitivity and for capture or binding of a single analyte at an aperture, the apertures are of micron order of dimension. The substrate and aperture array defines a microaperture array.

In the preferred exemplary arrangement, the substrate 120 is a polymer substrate or a substrate of any plastics material. A substrate of a mylar material is used in a preferred arrangement. It will be appreciated that substrates of suitable alternative materials may also be provided. The substrate 120 preferably has a substantially planar and uniform form and the material/s of the substrate are selected to support such requirements.

Apertures 122 are functionalised to provide capture of a target analyte at an aperture and to provide attenuation light transmittable via said aperture when a target analyte is captured or bound. Apertures 122 may be functionalised with a ligand to provide capture of a target analyte at an aperture to attenuate light transmittable via said aperture.

The photodetector 110 comprises an array of pixels 115. The size of apertures 122 patterned on the substrate 120 may be selected or varied to match the size/arrangement of pixels 115 of the photodetector. Accordingly, the system 100 is configured to provide an aperture 122 functionalised for capture of a specific analyte, the aperture being associated with a pixel 115 of the photodetector 110. The photodetector 110 is configured to detect light 171 transmitted through the aperture array 121. In particular, the photodetector 110 is configured to detect attenuation of light transmitted through the aperture array as a sample 150 including a target analyte/s 151/152 is provided to the substrate.

In one arrangement, as described above attenuation of light is detected. Attenuation may be detected based on the optical signal 115. The photodetector system 110 may be configured to detect a change in the binary signal or detected light signal or optical signal. A change in the signal is detectable when a sample 150 is provided to the substrate in comparison with a base level or calibration signal.

The system 100 provides for the transduction and detection of capture of target analytes 151/152 or biochemical binding events on the surface of the substrate or biochip 120.

As noted above, in a preferred exemplary arrangement, the system 100 is configured to provide correspondence or mapping between apertures 122 of the aperture array 121 of the substrate 120 and pixels 115 of the photodetector 110.

Accordingly, in one arrangement, the photodetector is configurable to detect a change in light transmitted via the substrate to the photodetector after capture of analytes in comparison with the light transmitted before the sample was introduced. Detection at the photodetector is effectively of a binary signal. Detection at the photodetector comprises detection of an event at an aperture. In the detected signal, light is either transmitted via an aperture or attenuated at an aperture. The signal may be provided as an electronic signal.

Concentration of analytes 151/152 in a sample is determinable by detection and processing of the change in the signal/optical signal being indicative of the number of apertures 122 at which light transmission is attenuated.

The photodetector system further includes data processing means/data processor 180. The data processing means 180 may include thresholding means 181 for thresholding a detected optical signal 115 and data processing means 182. The data 185 outputted from the system 100 provides indication of presence of target analyte(s) and/or concentration of target analytes 151/152 in a sample 150.

The thresholding is judiciously selected and set to allow for detection of analytes with minimum further data processing.

The microaperture array is configured to provide capture of specific analytes within a sample at an aperture. By making the biochip or substrate substantially optically transparent in only those regions where analytes can be captured and by placing it in close proximity to the photodetector, the present specification advantageously provides a system and method of transducing these so-called “capture events” into an electronic signal for analysis.

The system 100 is advantageously configured to detect a binary change in optical signal 115 based on detection of light transmitted via the substrate 120 to the photodetector 110. The system 100 advantageously comprises a lensless photodetector system. The system 100 is a compact relatively low cost system configured for ease of use.

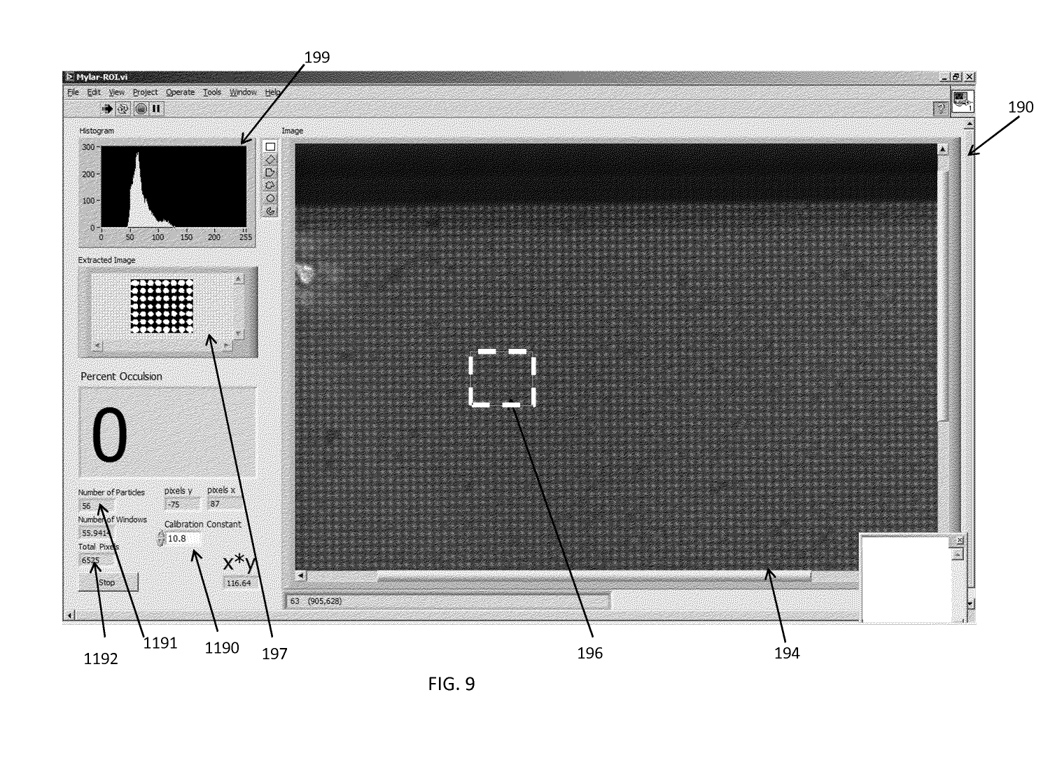

It will be appreciated that while the above system and method is based on detection at the photodetector of an optical signal which is further processed to provide result data, that in an alternative arrangement, detection may include capture of an image of the substrate at a photodetector. An exemplary arrangement of such an alternative system providing for capture and processing of images is described further below with reference to FIG. 9.

Referring to the drawings and in particular, FIGS. 1C and 1D, schematic images of an exemplary substrate 120 having an aperture array 121 of apertures 122 of the present specification are shown. Substrate 120 comprises a substantially non-transparent substrate patterned with an array of light transmitting apertures 122. The apertures 122 are functionalised or configured to provide capture of a target analyte 151/152 (as described above) at an aperture 122. As noted above the apertures 122 may be configured or functionalised in various ways to provide capture. In a preferred arrangement, apertures may be functionalised with a ligand to provide capture of a target analyte at an aperture to attenuate light transmittable via said aperture. Referring to FIG. 1D the effect capture of an analyte at an aperture 122 is illustrated as a blocked aperture 123. The blocked or partially blocked aperture 123 indicates a capture event or a binding event. As a result of capture of an analyte light at an aperture is attenuated or blocked is illustrated in the image.

The substrate 120 patterned with the array 121 of apertures 122 defines a photomask 125, wherein light is transmittable only through said apertures 122.

For example, a substrate of an exemplary arrangement of the present specification of FIG. 1C comprises an array of apertures having a diameter of the order of 5-30 microns (area of the order of 19 to 707 microns²). In a preferred arrangement, apertures having diameter of the order 10-20 microns (area of the order of 78 to 314 microns²). In a most preferred arrangement, the size of an aperture 122 is optimised to provide capture of a single target analyte at an aperture. The substrate 120 is thus configured such that the presence of a captured analyte is detected by detecting attenuation of light at an aperture.

The present specification further provides a method for detecting a target analyte.

The method includes one or more steps as follows:
Providing a substrate having an aperture array and wherein the apertures are functionalised for capture of a target analyte;
Providing a sample to the substrate comprising an aperture array
illuminating or passing light through the substrate to a photodetector,
Detecting light transmitted via the aperture array to the photodetector, (light transmitted is detectable as an optical signal/electronic signal), wherein the presence of an analyte captured at an aperture is detected in the signal detected at the photodetector.

The method further includes:
detecting a change in optical signal transmitted via the substrate before and after the sample is provided to the substrate.

Detecting a change in optical signal transmitted via the substrate before and after the sample is provided to the substrate may include a general calibration measurement or a number of measurements of optical signal during the course of a method of detection.

The method further provides for
processing of the optical signals as required to provide an indication of concentration of an analyte in a sample.

The method also provides
for multiplex detection of different target analytes in a sample. The substrate may be provided or configured such that different apertures are configured for capture of different analytes.

The substrate and system may be configured to provide detection of presence of analytes or for detection of concentrations.

The method also provides
capture of an image(s) of the aperture array and processing of the image.

The method also provides
real-time monitoring and real time processing of binding events at the aperture array.

In the arrangements of the present specification, the limit of detection is related to the size of the apertures 122 and the ability of the system 100 to capture a target analyte 151, 152 at an aperture.

Control and selection of the aperture size facilitates quantitative monitoring of an analyte or analytes 151, 152 in a sample. The arrangement of the present specification further provides for the monitoring of concentration of a target analyte in a sample.

The binary change in optical signal that is detectable is advantageously less dependent on detector characteristics such as sensitivity and noise since quantisation of a range of optical signals is not required than in other detection systems, than other signal types typically used in detection systems.

Consequently, the detection of single binding events (i.e. of single analytes 151,152) is possible using a system according to the present specification.

The binary nature of this transduction process and of the detection is a significant departure from the prior art and provides clear advantages.

For example, in one current approach, the intensity of an optical signal can be related to the concentration of analytes captured (i.e. fluorescently labelled molecules). In such systems, the limit of detection (LOD), which is related to the lowest intensity that can be reliably detected by the system, is dictated by the characteristics of the optical detector being used (e.g. sensitivity, noise). In the approach of the present specification, this is not the case.

The system of the present specification advantageously provides a reduced cost and label-free optical detection platform for the detection and quantitative monitoring of analytes in a sample.

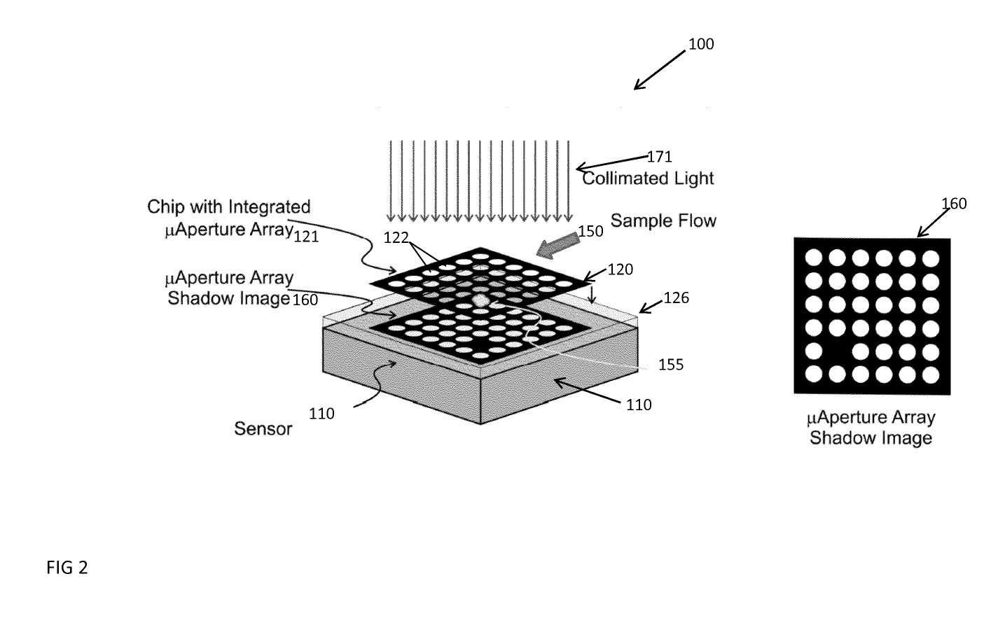

Referring to the drawings and in particular FIG. 1 and FIG. 2 an exemplary arrangement of the substrate 120 of the system 100 according to an embodiment of the present specification is described. The exemplary system 100 comprises substrate 120 which is a polymer (e.g. Mylar) substrate. The substrate 120 comprises an array of microapertures 122 which have been patterned in an array (μAA) to form microaperture array (μAA) 121. In the exemplary arrangement the apertures are transparent. The microaperture array 130 is placed directly on or close to the surface of photodetector 170 (e.g. CMOS or CCD image sensor). The microaperture array 121 and substrate 120 form a photomask 125 which blocks light from arriving at all the pixels 115 of the sensor 110 other than those directly beneath the apertures 122 of the substrate 120. In this configuration, an image 160 of the photomask 125 can be formed on the photodetector 110 by illuminating the μAA 122 with a suitable light source 170 located above the μAA. The image 160 is formed without the use of lenses. The nature of the lensless optical configuration of system 100 according to the present specification means that it is possible to have an aperture 122, functionalised for a specific analyte 151/152 associated with each pixel 115 of the image sensor 110. The size of the apertures 122 patterned on the substrate 120 to form the μAA 121 can be varied to match the pixel size of a sensor 115.

Referring to the drawings and in particular FIG. 2, an exemplary arrangement of an embodiment according to the present specification is described.

The system 100 comprises a substrate 120 configured for the capture of a target analyte 151 of a sample 150. The system 100 further comprises a photodetector 110. Light is transmittable from a light source 170 to the detector 110 via the substrate 120. The presence of an analyte captured at the substrate 120 is detected in an optical signal captured at the sensor 110. For the purposes of illustration of the operation of the system according to an embodiment of the invention, an image 160 of the substrate 120 as captured at the sensor 110 is shown.

The system 200 comprises a substrate 210 on which a plurality of apertures 222 have been patterned. In the preferred exemplary arrangement, the substrate is a non-transparent substrate and the apertures 222 are substantially transparent apertures. The apertures 122 have a diameter of the order of microns. The substrate 120 is patterned with an array of microapertures 122 providing a microaperture array (μAA) 121. The apertures 122 are configured to provide capture of a target analyte 151 of a sample 150 to be tested at an aperture. Each of the microapertures 122 is functionalised with a suitably selected molecule or ligand 200 (such as proteins, antibodies, aptamers, etc.) for the capture of a selected target analyte 151 (e.g. functionalised beads, molecule, cell, bacteria, virus) present in a sample 150 under test. Different apertures 122 may be functionalised for capture of different target analytes if appropriate allowing for a multiplex detection.

When a target analyte 151 is captured at an aperture 122, the amount of light passing through that aperture 122 will be attenuated (including completely blocked) resulting in a reduction in the number of apertures 122 detected by the photodetector 110. An aperture at which light is attenuated is effectively a partially or wholly filled or blocked aperture 123 and is detectable as such.

In the exemplary arrangement, the photodetector 110 is an array photodetector. The photodetector 110 may be for example a CMOS or CCD image sensor. The photodetector includes a plurality of pixels 115. The pixels 115 of the photodetector are provided as a pixel array.

The size of these microapertures 122 can be optimized to facilitate capture of a single analyte 151.

As shown in FIG. 1A or 2 in use, the substrate 120 is placed close to or directly on the surface of photodetector 110. For example, a spacer plate of glass 126 or other suitable material may be provided. Light 171 from the light source 170 is transmitted via the substrate 120 to the photodetector 110. The patterned substrate 120 defines a photomask 125 which blocks light 171 from arriving at all the pixels 115 of the sensor 110 other than those directly beneath apertures 122 of the substrate 120.

In one arrangement the optical signal of light transmitted via the substrate to the photodetector is captured. It will be appreciated that with a different arrangement of the photodetector, it is also possible to capture an image of the substrate. For the purposes of illustrating the operation and method of the invention, an image 160 of the photomask 125 that may be captured by an image sensor or at the photodetector 110 without the use of lenses is shown. However, it will be appreciated that as described above the system 100 is configured such that it is not necessary to capture an image of the photomask to detect a target analyte. Exemplary images of patterned substrates 120 captured at the photodetector are shown for example in FIGS. 2, 3, 5 and 6 discussed in further detail below.

The presence of an analyte 151 captured at an aperture 122 is detectable in the exemplary image 160 of the aperture array 125 captured at a sensor 110.

The arrangement of the present specification provides for detection of the optical signal transmitted via the substrate or in some cases even for the capture of an image of the substrate.

When an analyte 151 is present light at an aperture of the array is attenuated or blocked in comparison with the signal or image of the array prior to providing the sample to the substrate. The optical signal for detection is in a preferred arrangement a binary signal. A binary change in the optical signal is detectable.

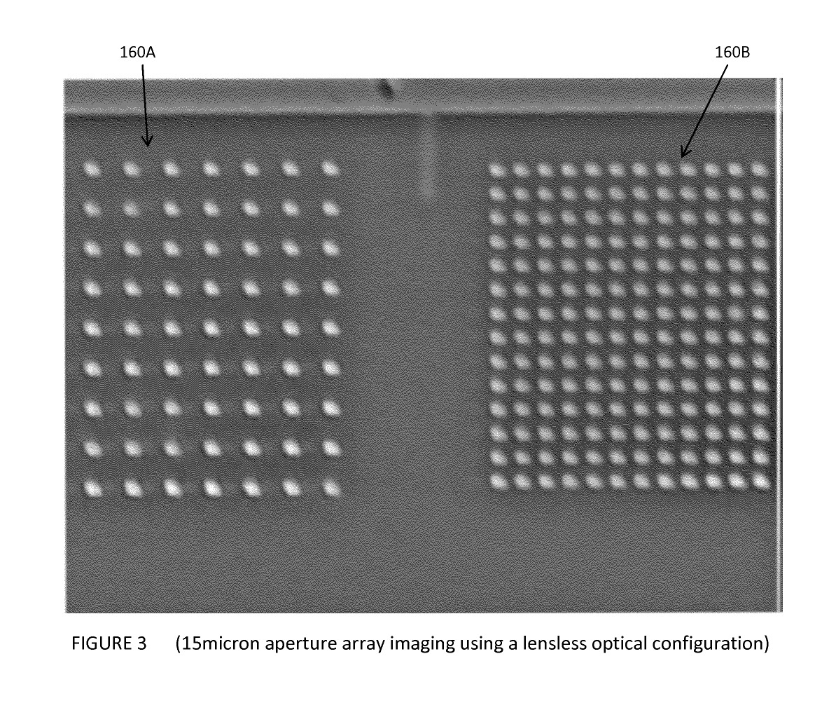

FIG. 3 shows images 160A and 160B of two example arrays 120A and 120B according to the present specification comprising 15 μm apertures 122 acquired using an optical system 100 according to an embodiment of the present specification.

Each of the apertures 122 in the μAA 120 may be functionalised with suitably selected molecules 200 (such as proteins, antibodies, aptamers, etc.) for the capture of a selected target analyte 151 (e.g. functionalised beads, molecule, cell, bacteria, virus) present in a sample 150 under test. Using an optical configuration such as that described above, when a target analyte 151 is captured at a microaperture 122, the amount of light passing through that aperture will be attenuated (including completely blocked) resulting in a reduction in light received by the pixels 115 located below the aperture 122. FIG. 3 illustrates schematically this concept.

Using the system 100 the concentration of analytes 151/152 in a sample 150 can then be determined by counting the number of occluded apertures 123 in an image 160 of the aperture array. FIGS. 4 (a)-(c) illustrate schematically a method of detecting analytes 151/152 in a sample 150 using a system 100 according to an embodiment of the specification as a sample 150 flows over a μAA 120.

Referring to FIG. 4 images showing the relationship between capture of analytes and an array 120 and image 160 of the array 120 obtained using an exemplary system 100 according to the present specification are shown. As sample 150 flows over the μAA 121 more analytes 151/152 are captured occupying more apertures 123 in the array 121.

Once captured, these analytes 151/152 reduce (or block completely) light arriving at the pixels 115 of the sensor 110 below the substrate 120 or array 121 causing an increasing number of apertures 122 to be blocked apertures 123.

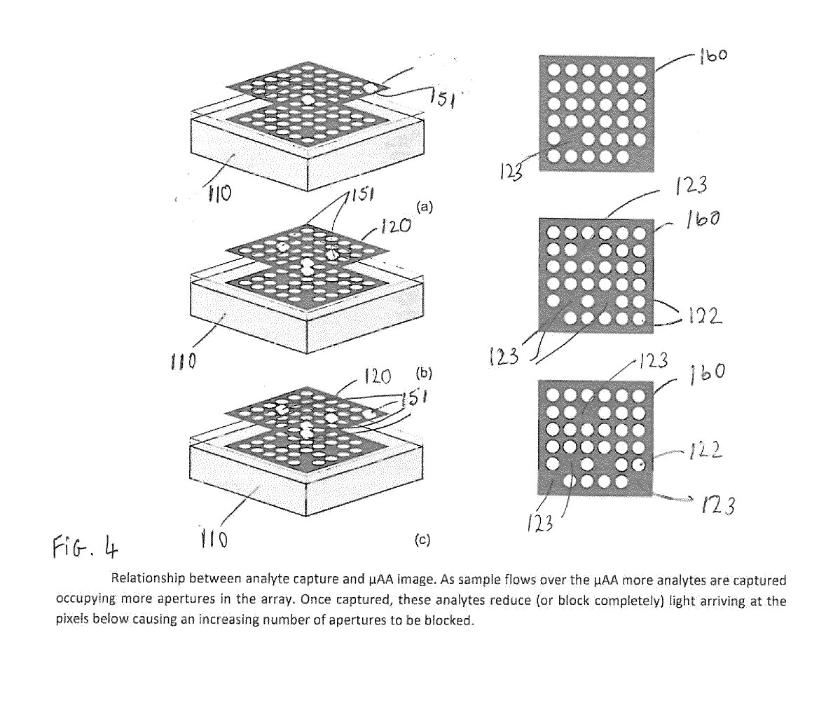

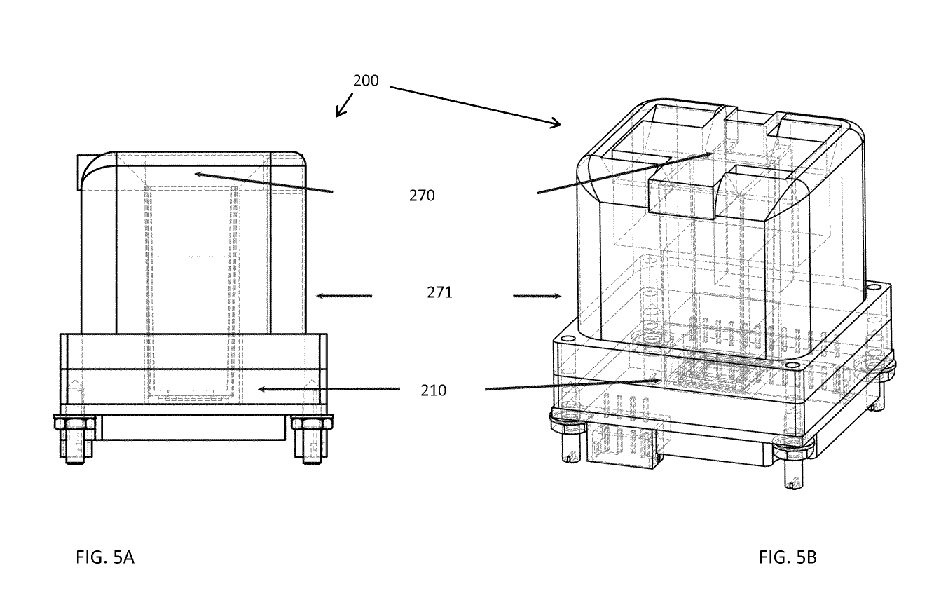

For example, an exemplary system 200 according to an embodiment of the specification is shown in FIG. 5. The system 200, of FIG. 5, comprises a CMOS image sensor 210 (from a webcam) and an LED light source 270. The system comprises a housing 271.

The system 200 is a lensless image system. The system is advantageously low cost to provide and maintain and compact for use at point of case. The sample is inserted into a receiver in the housing for imaging. Advantageously, the system is configured for each of use by the end user. The end user does not have to maintain the optical arrangement or adjust the optical arrangement. The housing preferably includes a slot for receiving the array between illumination of detector. The array may be imaged as binding occurs, as discussed below. The intensity may be measured as discussed above.

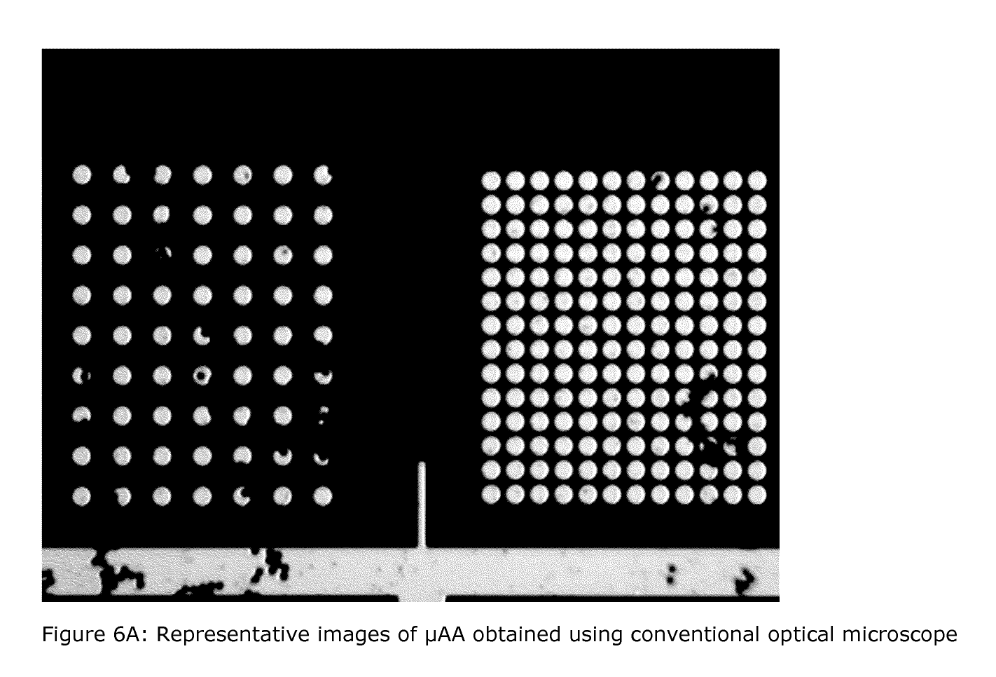

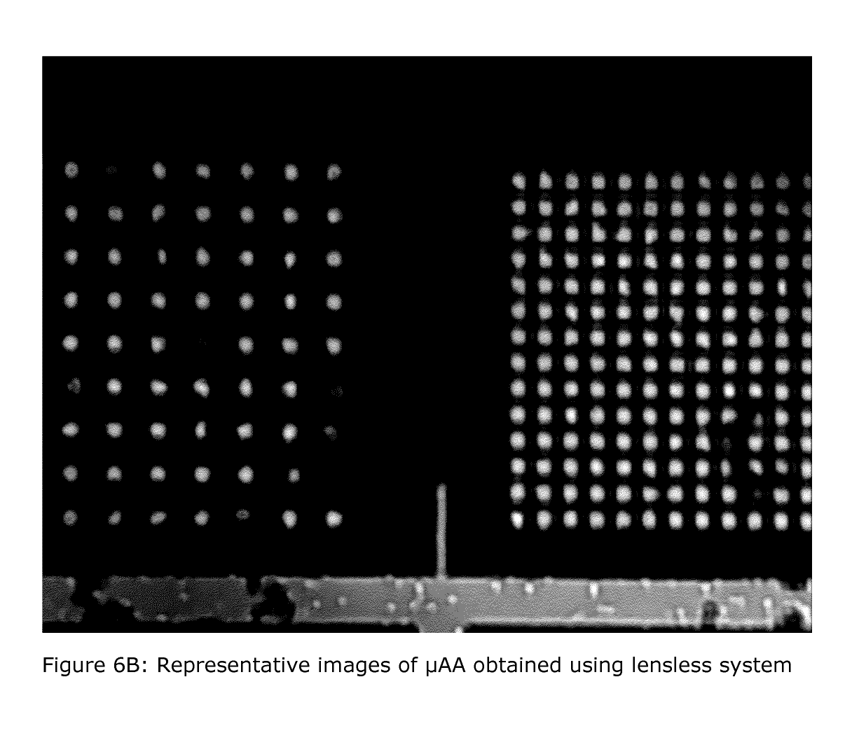

Referring to FIG. 6 results obtained by operation of the exemplary system 200 of FIG. 5 are shown. In this case, polystyrene beads 605 are provided and used to block a number of apertures 122 in the μAA 121. Polystyrene beads 605 in water solution were deposited on a Mylar sheet containing two patterned μAAs 121, 121′ to show detection of occlusion of apertures 122, 122′ using a lensless system according to the specification.

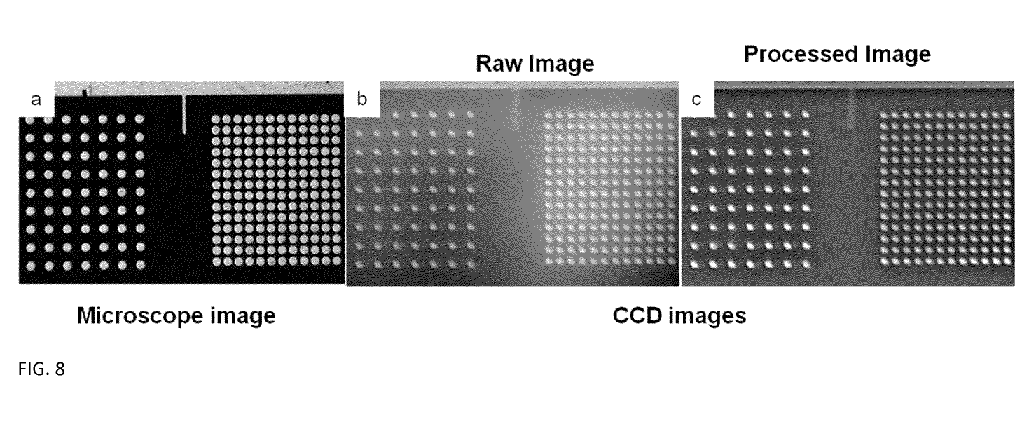

The images of FIGS. 6(a) and 6(b) show the same μAA 121 imaged using a conventional optical microscope and using an exemplary lensless system 100 according to the present specification, respectively. Reference is made also to FIG. 8 which show a series of images of a μAA taken by an optical microscope (a) and taken by the CCD sensor (b, c). Image (b) is the raw image, and (c) is the same image after it has been processed to show better contrast. This is an empty microaperture array (μAA). These figures of the exemplary arrangement show that the images collected by the CCD and an optical microscope are equivalent, and therefore the whole microscope is not necessary in the application shown.

There is clear correspondence between the two images indicating that i) the presence of beads 605 at apertures 122/122′ produces an occlusion of the aperture and ii) this occlusion can be detected optically using the lensless system.

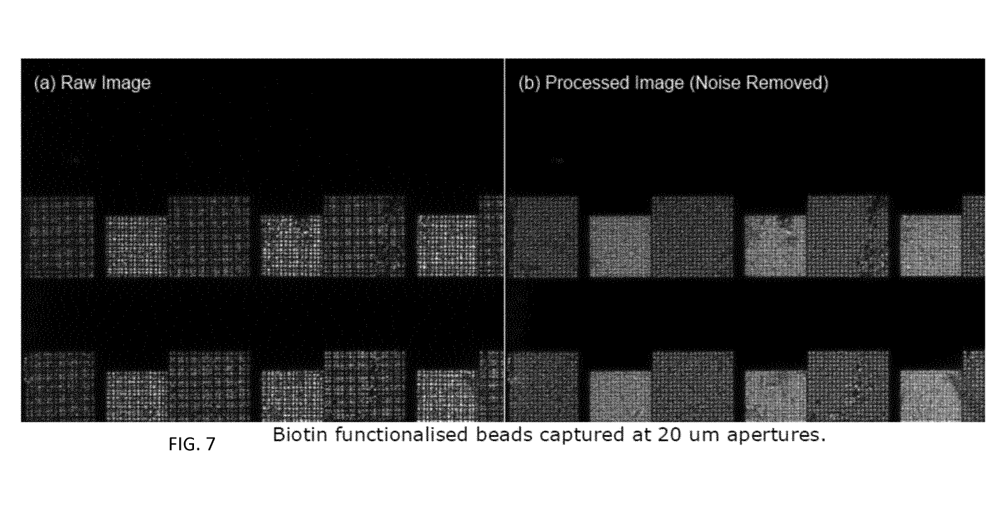

In a further example of operation of an exemplary system 100, biotin functionalised beads 705 were then used in a similar arrangement to demonstrate that the same detection process could be used where the beads 705 were captured on the surface of the array as part of a biorecognition event. FIG. 7 shows a typical image obtained.

The nature of the lensless optical configuration of system 100 according to the present specification means that it is possible to have an aperture 122, functionalised for a specific analyte 151, associated with each pixel 115 of the photodetector 110.

The size of the apertures 125 patterned on the substrate 110 to form the μAA 135 can be varied to match the pixel size of a sensor 150.

For example, FIG. 6 shows images of exemplary arrays 135 according to the present specification made up from apertures 125 that are substantially 20 μm in diameter. Clearly, a large portion of the field of view is unoccupied. Nonetheless, in this relatively suboptimal configuration it is evident that a large number of apertures are present. However, the image sensor used in this experiment contains 1290×960 pixels each of size 3.75 μm². In this case, it is possible for the field of view to contain over 1.2 million (1290×960=1,228,800) individual detection were the apertures reduced to 3.75 μm². This highlights a significant advantage of the lensless approach—the ability to combine a large sensor area with relatively high spatial resolution which in this case facilitates massive multiplexing capacity at potentially very low cost. Furthermore, since the system does not use any lenses, the image does not contain any artefacts associated with lenses such as vignetting or distortion.

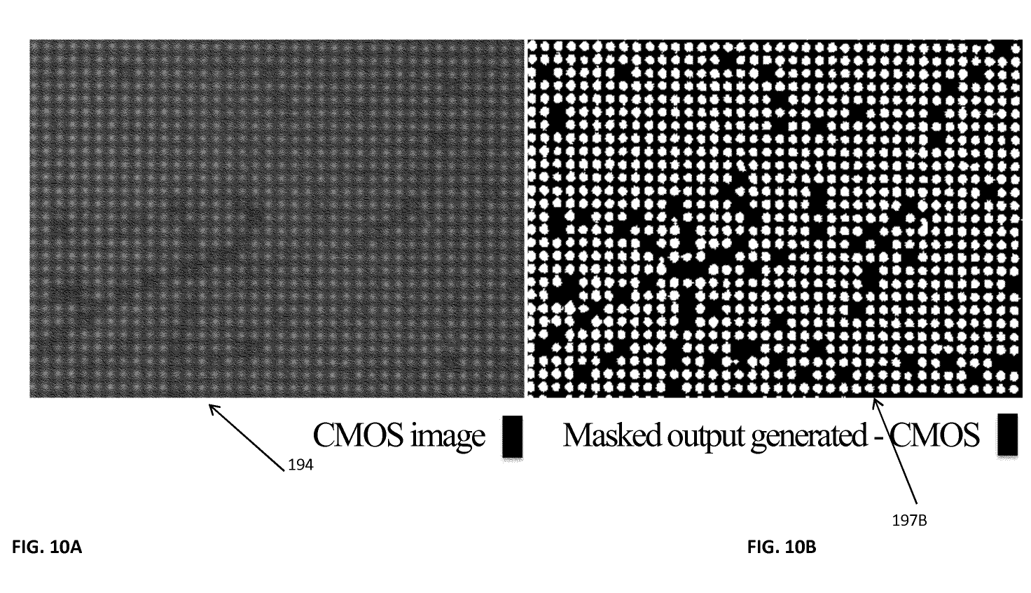

Referring to FIGS. 9 and 10 an exemplary data capturing and processing system 190 comprising a data acquisition module 191 and post-processing module 192 for obtaining data and interrogating said data according to the present specification is shown. The data may be image data. An exemplary interface 193 of the data-processing system 190 is shown. The interface shows exemplary image data 194 which is an image of an aperture array.

The system 190 may further comprise real-time monitoring means and data processing means 195 for ‘real-time’ analysis of binding events. Using this arrangement the system provides for the end-user to monitor binding and determine for example, percentage binding, inclusive of kinetic measurements as these binding interactions occur.

The processing system 190 provides a computer implemented method for the monitoring, capturing and processing of binding events, including in real-time.

The real-time monitoring and processing means 195 is configured to monitor and process data from binding events essentially as they occur. This allows for the analysis of data including relating to for example, binding rates, how fast binding occurs and how stable a particular binding event or analyte is.

In one exemplary arrangement, measurements may be performed in a single series of binding events. In another exemplary arrangement, parallel analysis on multiple arrays may be performed.

As illustrated the system 190 provides an adjustable grid 196 configured to be overlaid across the entire image or a portion thereof. The grid allows for selection and extraction of an image 197. Processing provides determination including the ‘percentage occlusion/occupancy’ for the area of interest as selected using the grid 196. Further, the grid 196 is configured to be adjustable to focus on a region of interest.

The exemplary arrangement provides a stand-alone system that is compatible with any webcam, and has been used in conjunction with a CMOS (Complementary Metal Oxide Semiconductor) imager for this preliminary analysis.

The system may further include a histogram profile means 199 to inform the end-user that the image is of sufficient quality to perform analysis.

The system may further include a calibration constant 1190 comprising an integral quality control feature. The calibration constant 1190 is determined prior to acquiring images to positively determine that the sensor imager is correctly calibrated to acquire and process images. As the Mylar pattern has a regular grid of apertures it can be deduced for any pixel dimension the number of aperture that exist in a specific region of interest. To aid in the calculation the calibration constant is used to determine the theoretical number of apertures. This theoretical value is then used in conjunction with the observed number of occluded apertures to determine a percentage occlusion value. Finally, the calibration constant can be applied for quality control purposes e.g. scratches, dust and printing, of the surface before commencement of the assay.

It has already been noted above that the exemplary system 180 provides for the capture of optical signal data and the exemplary system 190 provides for capture of image data, and for the processing of the image data.

As also noted above, a relationship may be provided between the number of pixels of the detector and the apertures of the array. Each aperture may relate to a number of pixels.

Binding events may be detected based on the attenuation of the light transmitted at apertures of an array. In addition or alternatively, binding events may be related to attenuation detected at specified of the pixels related to an aperture 122.

Referring to the exemplary arrangement of FIG. 9, it is noted that the grid 196 provides an extracted image 197 which relates to a particular number of apertures 1191 and pixels 1192. For example, FIG. 9 shows and exemplary grid and image that in the case illustrated relates to 56 apertures and 6525 pixels.

The system 190 accordingly provides that the data captured may be interrogated and processed essentially at a per aperture level. The system 190 accordingly provides that the data captured may be interrogated and processed based on the pixels involved, i.e. on a per pixel or a number of pixels related to an aperture.

Clearly, the data relating to attenuation at apertures or pixels may be used separately or in combination. The pixel level data for example, may be used to provide additional data relating to size or shape or form or any other suitable additional information.

Accordingly, the system provides a two-pronged approach or method, which can generate additional information about the mechanics of a binding event that is taking place on the surface. For example, it may be feasible to differentiate between particle clustering versus single binding of small particles. Percentage occlusion/occupancy is calculated on the assumption that the size of the occluded aperture is the significant parameter of interest. Percentage binding on the other hand takes a more granular approach looking at each pixel value in an aperture. If the intensity is above a pre-calculated threshold, the pixel is considered occluded relative to the other pixels in the image and a ratio value is determined. A histogram of the pixel intensity within all apertures is generated and a threshold is assigned, providing a ratio of occlusion to non-occlusion (e.g. percentage binding). Both approaches appear similar in nature but the subtle difference of intensity to size profile is enough to clearly make them key independent measurements.

Therefore, the system provides further for the combined use of two approaches; signal intensity drop and change in aperture shape. The existing system allows the detection of binding events using two approaches; the first approach relies on changes detected in the image of an aperture shape as a result of a binding event. In this instance, a binding event is detected by the software as a change in the image of an aperture shape. The second approach employs a decrease in signal intensity to detect a binding event.

It will be appreciated that the system provides for measurement of resultant binding/occlusion. A binding event resulting in occlusion at an aperture. The system as described provides for the occurrence of a binding event on the array and detection based on the consequence of the event, i.e. the presence of the particle or object which results in some blockage/attenuation of light.

Advantageously, the system is arranged to provide the imaging and data processing to determine binding events or concentration of analytes in a sample without the requirement for complex optics. There is no requirement for processing of diffraction data or other data pertaining to the interaction of light with the array and analyte. The system of the invention provides for determination of binding events at an aperture.

Referring to FIG. 10, the image processing steps of the exemplary system and method are described further. FIG. 10A (left hand image) shows an image that occurs resultant from binding events on the microaperture array, as detected by a CMOS image sensor. The generated output, shown in FIG. 10 B (the right hand image), is produced via a series of image processing steps, including for example: (1) image enhancement, (2) image thresholding, (3) image blob analysis and (4) quantification of binding (e.g. determining number of blocked apertures). The data generated from this analysis is then processed for example using the software of FIG. 9 to provide the end-user with a percentage occupancy/binding, in this case for an array of >1000 apertures.

The method includes one or more steps as follows:
Providing a substrate having an aperture array and wherein the apertures are functionalised for capture of a target analyte;
Providing a sample to the substrate comprising an aperture array
illuminating or passing light through the substrate to a photodetector,
Detecting light transmitted via the aperture array to the photodetector, (light transmitted is detectable as an optical signal/electronic signal/image/series of images of the aperture array), wherein the presence of an analyte captured at an aperture is detected in the signal and/or image detected at the photodetector.

The method further includes:
detecting a change in optical signal transmitted via the substrate before and after the sample is provided to the substrate.
   and/or
detecting a change in image of the substrate before and after the sample is provided to the substrate

Detecting a change in optical signal transmitted via the substrate before and after the sample is provided to the substrate may include a general calibration measurement or a number of measurements of optical signal during the course of a method of detection.

The method further provides for
processing of the optical signals/images as required to provide an indication of concentration of an analyte in a sample.

The method also provides
for multiplex detection of different target analytes in a sample. The substrate may be provided or configured such that different apertures are configured for capture of different analytes.

The substrate and system may be configured to provide detection of presence of analytes or for detection of concentrations.

The method may also provide
capture of an image(s) of the aperture array and processing of the image.

The method may also provide
real-time monitoring and real time processing of binding events at the aperture array.

The method may also provide
processing of spatial information e.g. processing of image data to determine which apertures are occluded and the extent of occlusion of an aperture e.g. to determine further information about the type of binding event occurring.

In experimental setup, apertures of 20 μm (in diameter) were provided together with streptavidin-labelled magnetic beads (15 μm in diameter) which bind to different concentrations of biotinylated horseradish peroxidase (HRP).

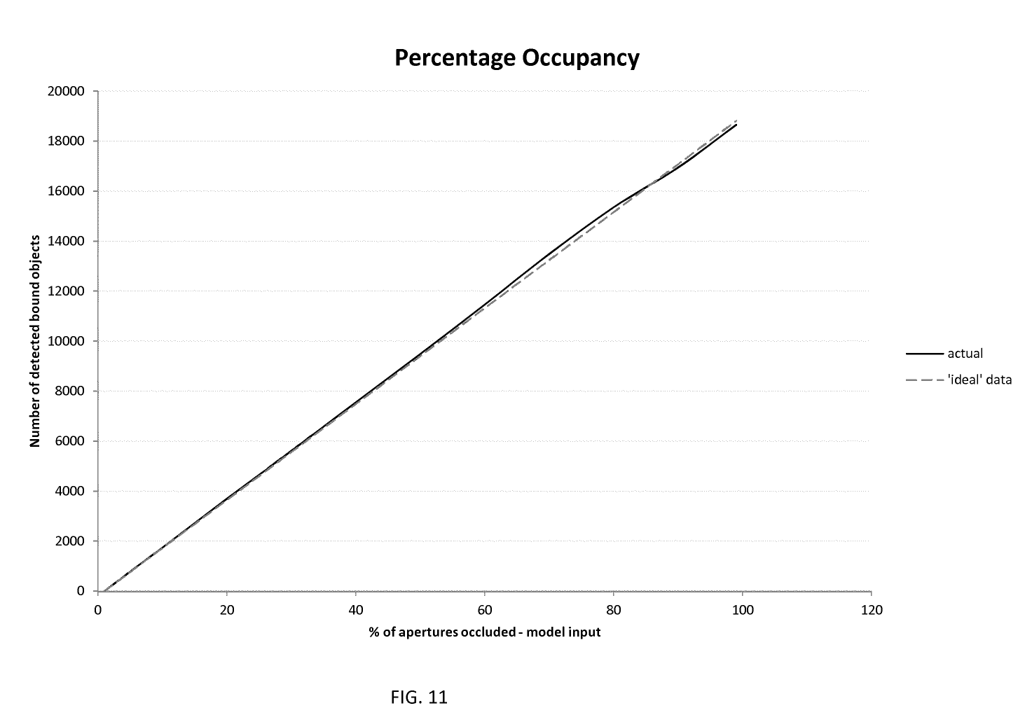

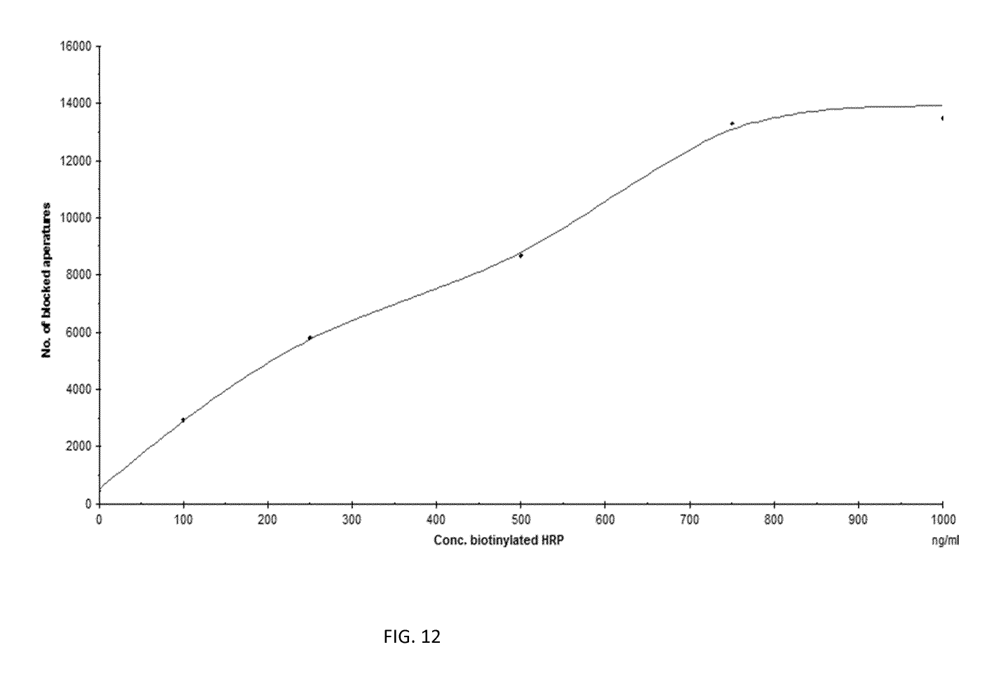

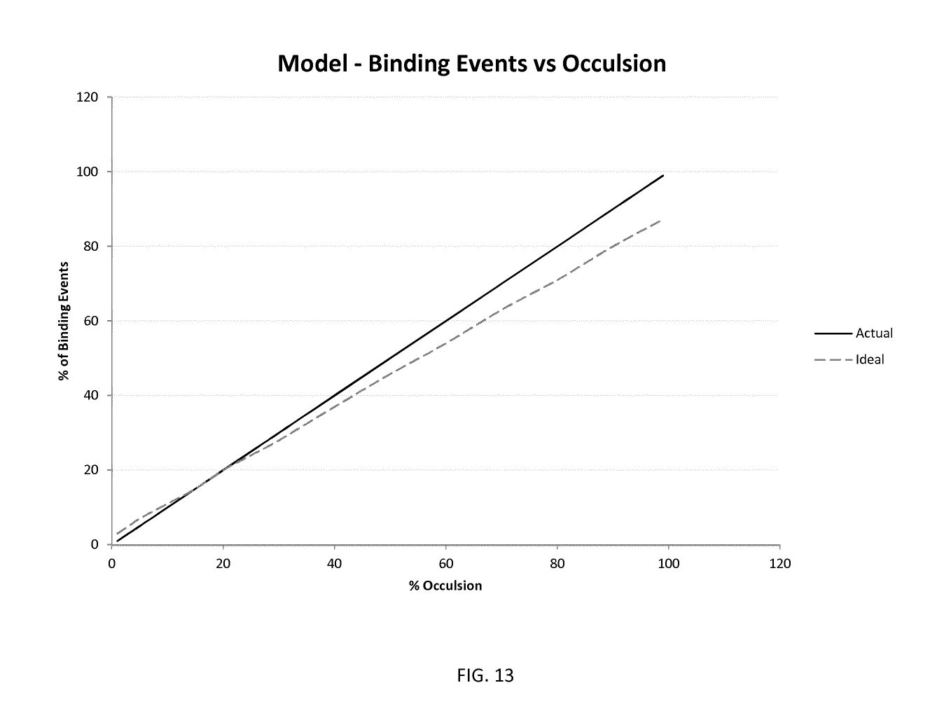

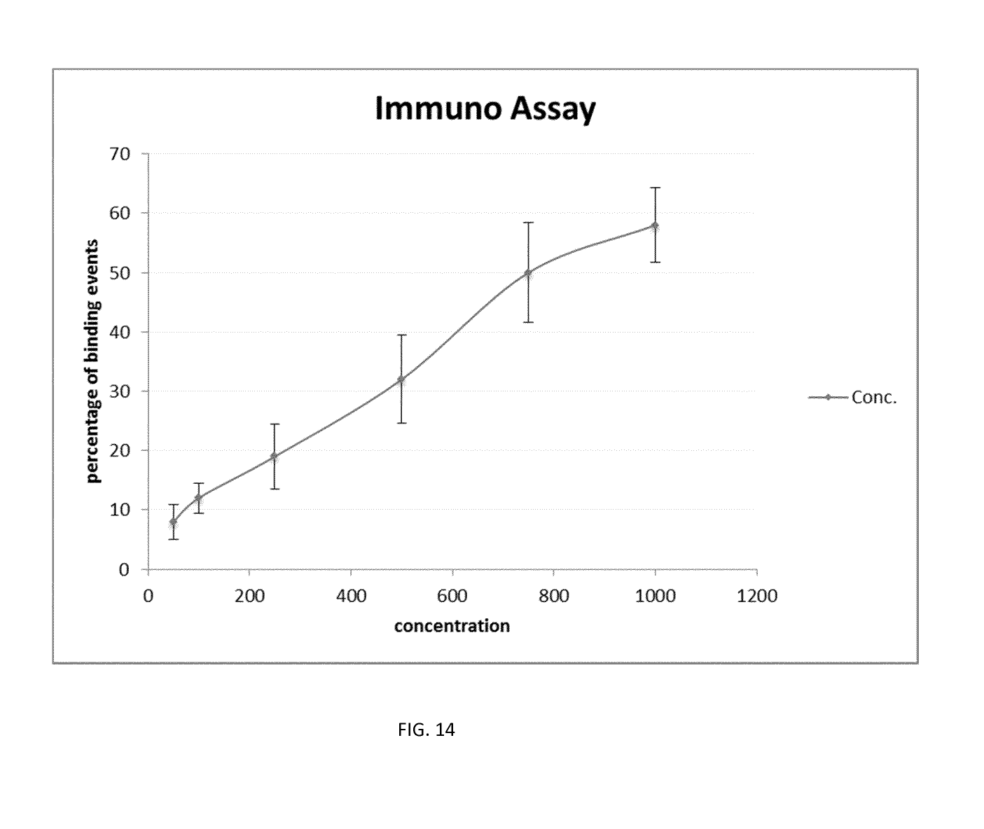

FIGS. 11 to 14 provide graphs demonstrating various results that may be determined using a Microaperture Array System according to the present specification to detect and evaluate binding events.

The approach of the invention has a number of advantages over the prior art. For example, in the case of the system of the specification no attempt to acquire an image of the analyte is made. The microaperture array 121 provides a means of capturing specific analytes 155 within a sample 150. By making it optically transparent in only those regions where analytes can be captured and by placing it in close proximity to the photodetector, the system and method of the present specification provides means for transducing these “capture events” into an electronic signal for analysis.

In a further arrangement and/or application, the method may include:
staining or labelling the cell, protein, or nucleic acid fragment using a “generic” stain, dye, chromophore or nanoparticle, i.e. one that is not specific to a particular species, strain, protein, or sequence

The method may further or alternatively include
selecting the wavelength of the illuminating LED/LD/etc. so that it is at or near the absorbance maximum of the stain/dye/chromophore/nanoparticle.

The above steps enhance the limit of detection, which in some cases could improve by one to several orders of magnitude (e.g. the amount of light occluded by a single small protein is not likely to be detectable without dye).

The approach noted, to label or dye a target species using non-specific labels is advantageously applied with ease within the context of the method and system described. It does not require that users develop custom antibodies or nucleic acid probe sequences that bind only with a single target. In packaging a system for commercial use, there would not be the need to have a different “version” of the labelling reagents for every different target. The specific recognition feature on the chip itself would be needed.

In a further alternative arrangement specific labelling may be used. For example, specific labelling may be used to provide option of a sandwich assays best, involving two separate independent recognition steps of the same target, and this can decrease non-specific responses (false positives) for example depending assay context, target, and environment.

The arrangement of the specification advantageously obviates the need for extensive image processing. There is no requirement for image processing algorithms.

The microaperture array provides a means of capturing specific analytes within a sample. By making it optically transparent in only those regions where analytes can be captured and by placing it in close proximity to the photodetector, the present specification advantageously provides a system and method of transducing these so-called “capture events” into an electronic signal for analysis.

The system according to the present specification advantageously constitutes the basis for a platform technology, a “universal reader” for analytical assays based on surface binding events.

Advantageously, the use of a system according to the present specification combined with degas driven flow microfluidics (a family of self-powered microfluidic devices) provides an arrangement comparable to the use of lateral flow assays, however offering higher sensitivity and accuracy.

The system according to the present specification advantageously is provided for integration for the development of a Point Of Care (POC) test for Platelet Function to monitor the effect of antiplatelet drugs.

Advantageously, the approach of the present specification makes use of a specialised optical detection setup which does not require use a microscope or complex optics and thus provides application outside of the inherent magnification/field of view limitations. It will be appreciated that a requirement for a microscope or complex optics typically limits application or use of a particular detection system to a laboratory. When imaging an array a trade-off is made between the spatial resolution of the optical system and the sampling of the image that can be provided by the image sensor, for example, with regard to magnification and field of view. The method and system of the present application in contrast provides detection of a statistically significant number of binding events on an array of apertures without the use of lenses. This approach is advantageously simpler than a system using a microscope and the synergy between the array detection system and the imaging system advantageously provides improved results while addressing issues with complex optics. The system does not require use of lenses. However, as discussed in the present specification and demonstrated in the graphs of data provided, the system can be used to capture spatial information without the use of lenses.

Advantageously, the present arrangement provides for an array geometry that is not dependent on the incident radiation or having a limitation to use of small size of the aperture so as to manipulate or interact with light waves as they pass or attempt to pass through. In the prior art, system may have relied on diffraction phenomena. In contrast the present application provides for simple transmission of light.

In prior art approaches, apertures may be metal and the material in the apertures may be a “sensing material” meaning that it undergoes a change of some sort that is detectable. In contrast, the present specification provides use of a binding/capture agent or moiety; In the prior art, apertures and spaces between them may be restricted to being of the order of or smaller than the wavelength of interrogating light, a consequence being significant diffractive effects. In contrast, the method of the present specification may provide apertures are greater than the wavelength of light. However, the method of the present specification is clearly not limited as such.

The method of the present specification is based on simple transmitted intensity with no frequency-specific or polarization-specific response required. The system does not rely for example on diffraction phenomena.

The method of the present application provides detection based on an occlusion of the light via selective binding of the target species in/over the apertures. The method is advantageously simplified relative to the prior art.

Advantageously the method of the present specification facilitates multiplexing. The present specification provides, that one substrate may be used for multiple analytes (as opposed to multiple substrates). This is achieved by virtue of the fact that the method and device of the present specification enables spatial information to be retained i.e. it is advantageously possible to interrogate specific analyte sensitive locations using system and single image sensor.

The method and device contrasts to spectroscopic data collected through a light aperture array. Advantageously, the μAA system can utilise any light source, and does not rely on spectroscopy (i.e. differentiation and examination of specific wavelengths, polarisation, plasmon effects) or any optics other than the CMOS sensor. While a system that uses diffractive phenomena as a basis for detection would require an ordered arrays, it will be appreciated that the system of the present application does not require provision of an ordered array to produce a diffraction output, various forms of array are envisaged and exemplary arrangements are noted above however, it will be appreciated that the array of the present application could also be random in form. The only requirement for the binding surface is that it is of sufficient transparency to enable sufficient light to pass through it, rather than contributing to optical effects e.g. the use of a metal film for plasmon effects.

Advantageously, the present application provides a system which may be arranged for multiplex detection. There is no requirement for multiple substrates to achieve multiplex detection. Advantageously, one substrate may be used for multiple analytes. Advantageously, the approach described enables spatial information to be retained i.e. it is possible to interrogate specific analyte sensitive locations using the system and the lensless image sensor. Further, the system provides multiplex detection using a single image sensor.

Accordingly, one of the core aspects of approach of the present specification is that spatial information can be retained without the use of lenses.

Advantageously, the substrate may be as described a mylar sheet. However, it will be appreciated any suitable substrate of a plastics or polymer material e.g. Zeonor may be used. This reduces the costs and fabrication time for fabrication of the substrate. The arrangement may also provide a recyclable substrate. The substrate material may be transparent and may be provided with a mask defining apertures to transmit light and defining non-transmitting portions between the apertures. In other approaches a photomask may have been used to form a pattern on another substrate.

It is noted that the μAA system of the present specification provides means for reading and detecting particles which are not metal and not necessarily completely opaque e.g. the present system and method provides for detection of platelets.

The present method and system provides an alternative to system that require optical complexity or additional steps e.g. metallisation of semi-transparent particles to facilitate polarisation/Plasmon effects, the device and method of the present application effective obviate these requirements.

The system according to the present specification further provided for integration with mobile technologies such as mobile phones, tablet, laptops, etc. advantageously having low energy power consumption, fast results. Such arrangement including the system integrated with a mobile device can be used for graphical interface of the results, remote monitoring.

The words comprises/comprising when used in this specification are to specify the presence of stated features, integers, steps or components but does not preclude the presence or addition of one or more other features, integers, steps, components or groups thereof.

1. An optical detection system comprising a photodetector and a substrate, wherein
the substrate comprises an aperture array wherein light is transmittable to the photodetector via the apertures of the substrate only, and
the apertures are functionalised to provide capture of a target analyte at an aperture such that capture of an analyte at an aperture causes attenuation of light at said aperture,
the photodetector being configured to detect the capture of an analyte at an aperture of the aperture array by detecting attenuation at an aperture,
wherein the system comprises a lensless detection system.

2. The system as claimed in claim 1, wherein the concentration of analytes in a sample is determinable based on detection of the number of apertures at which light transmitted is attenuated.

3. The system as claimed in claim 1, wherein the system is configured to detect a binary change in the detected signal of light transmitted via the substrate to the photodetector.

4. The system as claimed in claim 1, wherein the substrate comprises a substantially non-transparent substrate patterned with an array of substantially transparent apertures.

5. The system as claimed in claim 1, wherein the substrate comprising the aperture array defines a photomask, wherein light is transmittable only via said apertures of the substrate.

6. The system as claimed in claim 1 wherein the apertures have a diameter of the order of 5-30 microns.

7. The system as claimed in claim 1 wherein the size of an aperture is optimised to provide capture of a single target analyte at an aperture.

8. The system as claimed in claim 1 wherein the substrate comprises a polymer substrate.

9. The system as claimed in claim 1 wherein the substrate comprises a mylar substrate.

10. The system as claimed in claim 1 wherein the apertures are functionalised to provide capture of a target analyte by any suitable means including for example, chemical, mechanical, magnetic means.

11. The system as claimed in claim 1 wherein the apertures are functionalised with a ligand to provide capture of a target analyte at an aperture to attenuate light transmittable via said aperture.

12. The system as claimed in claim 1 wherein the apertures are functionalised by chemical patterning technique, such as ink jet printing, micro-contact printing, dip pen nanolithography, or light directed synthesis.

13. The system as claimed in claim 1 wherein different apertures of the array are functionalised to provide capture of different target analytes.

14. The system as claimed in claim 1 wherein the photodetector comprises an array of pixels.

15. The system as claimed in claim 14 wherein the size of apertures patterned on the substrate can be varied to match the size of pixels of the photodetector.

16. The system as claimed in claim 14 wherein the aperture array is configured to provide correspondence between apertures of the array and pixels of the photodetector.

17. The system as claimed in claim 1 wherein the aperture size is optimised to provide capture of a single target analyte at an aperture.

18. The system as claimed in claim 1 wherein the concentration of analytes in a sample is determinable based on detection of the number of apertures at which light transmission is attenuated.

19. The system as claimed in claim 1 wherein the system is configured to detect a change in the detected signal of light transmitted via the aperture array after capture of analytes.

20. The system as claimed in claim 1 wherein a target analyte for example, a cell or protein or nucleic acid fragment is stained or labelled using a stain or dye or chromopohore or nanoparticle.

21. The system as claimed in claim 1 wherein the light comprises light having predefined wavelength or wavelength range.

22. The system as claimed in claim 20 wherein the wavelength is selected to be at or near an absorbance maximum of the stain or dye or chromophore or nanoparticle.

23. The system as claimed in claim 1, wherein the intensity of an optical signal is related to the concentration of analytes captured.

24. The system as claimed in claim 1, wherein the system comprises data processing means.

25. The system as claimed in claim 1 wherein the data processing means comprises thresholding means.

26. The system as claimed in claim 1, wherein data processing comprises an image data acquisition means and image data interrogation means.

27. The system as claimed in claim 1, wherein the captured data comprises image data.

28. The system as claimed in claim 27 wherein the image data is processed to determine percentage binding and size profile information.

29. The system as claimed in claim 1, the data processing means comprising real-time monitoring means.

30. A substrate configured to provide detection of capture events on the surface thereof comprising a non-transparent substrate patterned with an array of transparent apertures wherein the apertures are functionalised to provide capture of a target analyte at an aperture, wherein
the substrate patterned with the array of apertures defines a photomask, light being transmittable only through said apertures,
the size of an aperture being optimised to provide capture of a single target analyte at an aperture,
the substrate comprising a polymer substrate.

31. The substrate as claimed in claim 30 wherein the apertures have a diameter of the order of 5-30 microns.

32. The substrate as claimed in claim 30 wherein the apertures are functionalised by chemical patterning technique, such as ink jet printing, micro-contact printing, dip pen nanolithography, or light directed synthesis.

33. A substrate as claimed in claim 30 wherein different apertures of the array are functionalised to provide capture of different target analytes.

34. A substrate as claimed in claim 30 wherein the apertures are functionalised with a ligand to provide capture of a target analyte at an aperture to attenuate light transmittable via said aperture.

35. A substrate as claimed in claim 30 wherein the presence of a captured analyte is detected by detecting attenuation of light at an aperture.

36. A substrate as claimed in claim 30 wherein the substrate comprises a biochip for use in the detection of biochemical binding events on the surface of a biochip.

37. A method for detecting the presence of a target analyte in a sample using a lensless optical detection system that comprises a photodetector and a substrate, wherein the substrate comprises an aperture array wherein light is transmittable to the photodetector via the apertures of the substrate only, the apertures are functionalised to provide capture of a target analyte at an aperture such that capture of the target analyte at respective ones of the apertures causes attenuation of light at said aperture, the photodetector optically positioned to detect the capture of the target analyte at the respective apertures of the aperture array by detecting the attenuation at the respective aperture, the method comprising:
providing a sample to the substrate,
passing light through the substrate to a photodetector, and
detecting light transmitted via the substrate to a photodetector,
wherein a change in the detected light indicates presence of a target analyte.

38. The method as claimed in claim 37 wherein light transmitted via the substrate to the photodetector is detectable as an optical signal/electronic signal.

39. The method as claimed in claim 37, comprising staining or dyeing a target analyte for example, a cell or protein or nucleic acid fragment using a stain or dye or chromopohore or nanoparticle.

40. The method as claimed in claim 37, wherein the light comprising light having predefined wavelength or wavelength range.

41. The method as claimed in claim 37, comprising selecting the wavelength of the light such that the light has wavelength at or near an absorbance maximum of the stain or dye or chromophore or nanoparticle.

42. The method as claimed in claim 37, comprising detecting the intensity of an optical signal wherein the intensity of the optical signal is related to the concentration of analytes captured.

43. The method as claimed in claim 37, further comprising
processing of the optical signal to provide an indication of concentration of an analyte in a sample.

44. The method as claimed in claim 37, further comprising
capturing an image of the aperture array.

45. The method as claimed in claim 44 further comprising
determining percentage of binding events in a sample.

46. The method as claimed in claim 44 further comprising
determining percentage occlusion/occupancy of an array.

47. The method as claimed in claim 37 further comprising
providing real-time monitoring of the array.
